(19) United States
(12) Patent Application Publication
(10) Pub. No.: US 2007/0043367 A1
(43) Pub. Date: Feb. 22, 2007

(54) Orthopaedic appliances

(76) Inventor: Steven Lawrie, Budderim Queensland (AU)

Correspondence Address: MICHAEL MOLINS;MOLINS & CO., SUITE 5, LEVEL 6, 139 MACQUARIE ST, SYDNEY NSW, 2000, AU

(21) Appl. No.: 11/188,019

(22) Filed: Jul. 25, 2005

(51) Int. Cl. A61F 2/30

(52) U.S. Cl. 606/69

(57) ABSTRACT

Provided is a bone plate for the olecranon of the proximal ulna using a combination of screws and K wires. The plate is curved to fit the shape of the olecranon and is screwed in place. The plate has a locking section located in the curved section. A fastener is pushed over the ends of the K wires and locks in to the locking section of the plate. The fastener has a tapered wedge that secures the K wires allowing fixation to the plate and resists their withdrawal. A femoral stem cap is used during hip replacement surgery and protects the Morse taper of the femoral stem once the ball of the hip joint is removed. The femoral stem cap has an air vent to allow air to pass when being applied and removed.

### FIELD OF THE INVENTION

The invention relates generally to orthopaedic appliances and more specifically to a plate and Kirshner-wire (K wire) system used for fixation of fractures of the olecranon. Also disclosed is a protective end cap for the femoral stem in hip replacement surgery.

### BACKGROUND AND RELATED ART

Certain types of fractures of the upper arm bones cannot be effectively healed without the use of a bone screw, pin, cable, wire or plate. The use of each of these is known to the prior art.

One of the most common fractures of the elbow involves the olecranon, the proximal end of the ulna. In the most typical fracture pattern, the olecranon is severed from the rest of the ulna along the bottom of the trochlear notch. Such fractures are often difficult to treat because the powerful muscles attached to the olecranon tend to pull it away from the rest of the ulna and rotate it around the trochlea. It is necessary to reattach the severed olecranon to the ulna to immobilize and stabilize the bone until healed.

Another common orthopaedic procedure is a hip replacement with a ball and socket artificial or prosthetic hip joint. It is common that an artificial or prosthetic hip joint needs servicing after several years of use. Sometimes the socket is replaced and not the ball. The ball in some replacement hip joints is held onto a stem by a friction fit called a “Morse taper”. When replacing the socket, the ball is removed from the femoral stem so it is not in the way during surgery. With the ball removed, the Morse taper is vulnerable to damage such as scratching. What is needed is a protective cover or cap for the femoral stem during hip replacement procedures.

### OBJECTS AND SUMMARY OF THE INVENTION

The olecranon plate is a segmented metal plate with holes to accept cooperating screws and K wires. The olecranon is fixed in place with two K wires that are held in place by a threadless friction type fastener that is applied once the plate and K wires are in place. The fastener prevents the K wires from backing out resulting in a loss of fixation and fixes the plate rigidly to the K wire.

It is one object of the present invention to provide an olecranon plate for the fixation of fractures of the elbow. It is another object to provide a plate that fixes the olecranon or other bone using K wires, and a threadless fastener that is applied after the K wires are in place, preventing the K wires from backing out of the plate, or advancing and thus gaining fixation to plate. It is another object to provide a plate that can be used for all fractures and osteotomies of the olecranon.

It is also an object to provide a threadless or friction type fastener that can be used to secure a K wire in a variety of circumstances.

The femoral cap is a disposable plastic cap shaped to provide a sterile protective cover for the femoral end during hip replacement surgery. It is placed over the femoral Morse taper after the ball is removed from the femoral stem. It remains protectively in place while revising the socket. A flange at the cap base allows the cap to be easily removed when time to reinsert the ball once the socket is revised. A vent in the top of the cap allows air to escape when the cap is fitted over the femoral stem. The cap has an internal diameter to fit tapers in common use.

The cap is also used to protect the Morse taper in primary hip replacements when protection is necessary such as in mini-invasive surgery.

### BRIEF DESCRIPTION OF THE DRAWINGS

FIG. 1 is a side elevation of an olecranon plate and olecranon

FIG. 2 is a perspective view of an olecranon plate with locking section

FIG. 3 is a top plan view of an olecranon plate with locking section

FIG. 4 is a perspective view of the K wires in place with the locking section

FIG. 4a is a cross section view of the K wires, locking section and fastener

FIG. 5 is a cross section view of the fastener engaging the K wires

FIG. 6 illustrates an inverted perspective view of the fastener

FIG. 7 is a perspective view of the olecranon plate

FIG. 8 illustrates a cross sectional view of the femoral stem cap

### DETAILED DESCRIPTION OF THE PREFERRED EMBODIMENTS

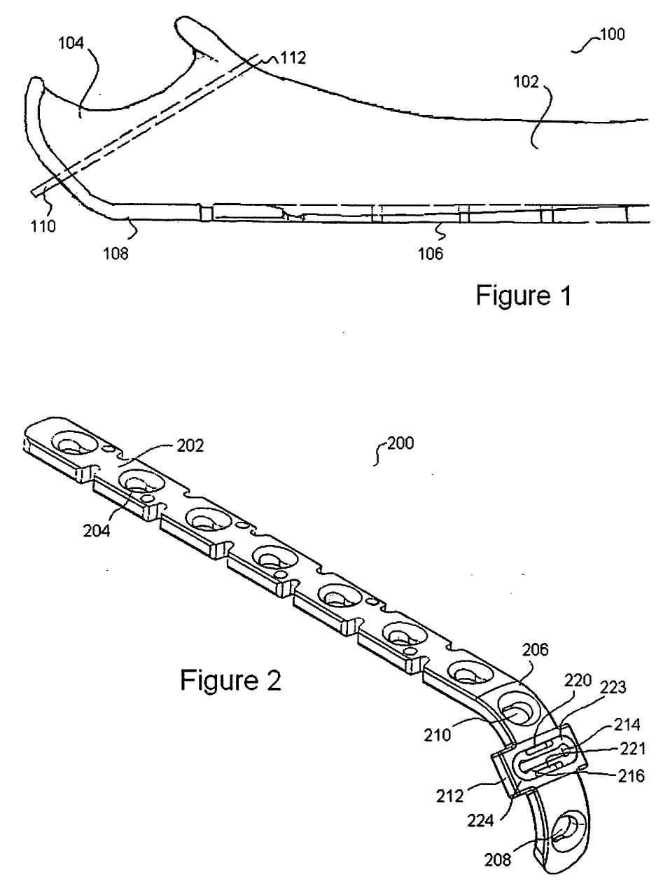

The bone plate of the present invention is adapted for fractures of the olecranon but the principles taught herein, particularly with regard to the fastening of K wires, may have application in a variety of K wire fixation systems. FIG. 1 illustrates the ulna bone 102 and olecranon 104. The olecranon plate 106 is attached to the ulnar shaft using screws (not shown). One end of the olecranon plate is bent to shape the curve of the ulna 108. One or more, and in this example two laterally spaced, parallel K wires 112 pass through the olecranon plate 110 and into the ulna.

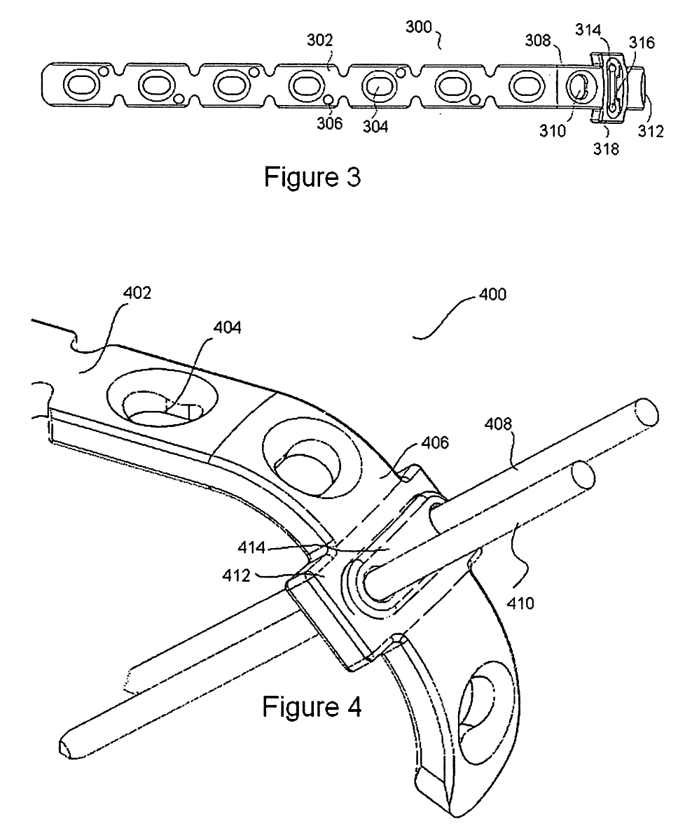

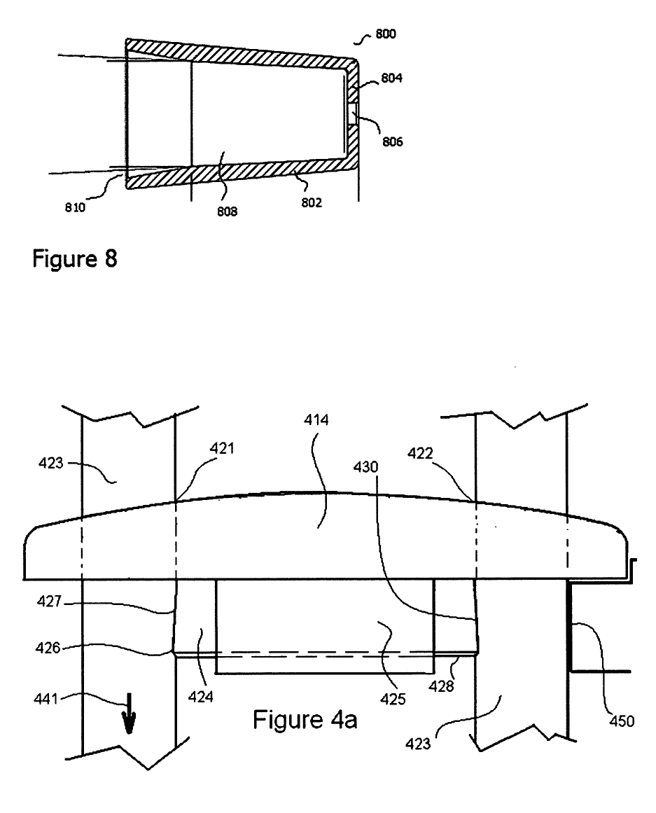

As shown in FIGS. 2 and 3, the plate comprises a notched or segmented shaft portion 202, with at one or more counter-bored shaft screw hole 204 for cooperating fixation screws. Opposing notches that define the segments allow the shaft to be bent, as required. The screw or screws are used to attach the plate to the shaft of the ulna. The plate 202 has a curved end 206 with two screw holes 208 and 210 that are used hold the curved portion against the curved section of the ulna. A rectangular locking section 212 lies, for example, between these last two screw holes and has a slot formed by two interconnected openings 214 and 216. In this example the locking section is wider than the remainder of the plate, providing optimum spacing for the K wires. The openings are for receiving the K wires. The slot also receives the wedge 424 of the fastener (see FIG. 4). The slot 214, 216 is flanked by a pair of generally parallel and rectangular openings 220, 221. The openings 220, 221 and the slot 214, 216 are contained within the sidewalls of shallow well 223 having rounded ends 224.

FIG. 4 illustrates the locking section 412 with K wires 408 and 410 and fastener 414 in place. The fastener is threadless and acts like a clamp, resisting the movement of the K wires through the fastener. The fastener 414 slides into position within the well in the locking section by pushing it over the ends of the K wires once they are in place. When the fastener is inserted fully and snaps into place, it engages permanently with the locking section. A fastener insertion tool 90 is shown in FIG. 9. The tool comprises a a shaft 91 with a handle 93 at one end and a head 92 at the other. The head has an underside that is contoured to cooperate with the upper surface of teh fastener. The head has two through holes 94 for receiving the K wires 95 (already in position). The shaft joins the head between the holes 91. The handle is used to grip the tool so that it can be used to drive and eventually tap the fastener into a “snap” locking engagement with the locking section.

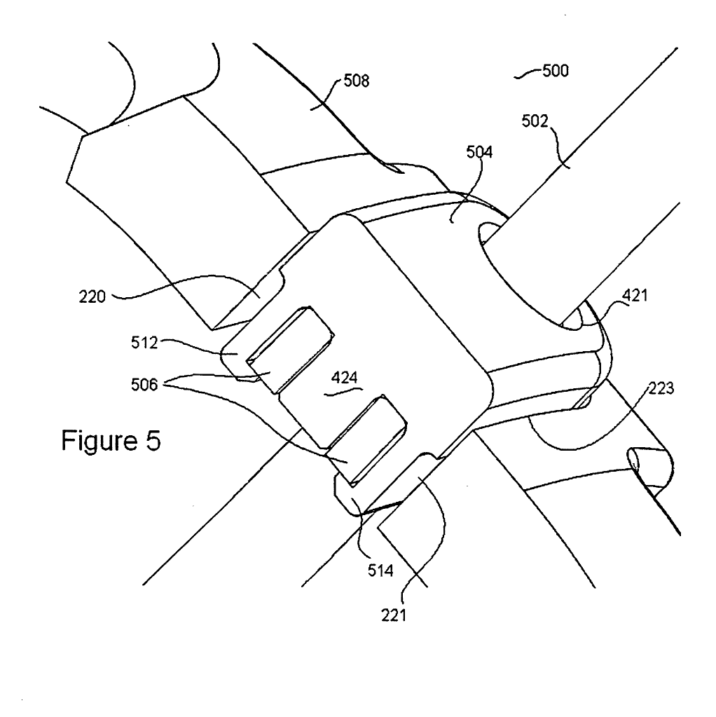

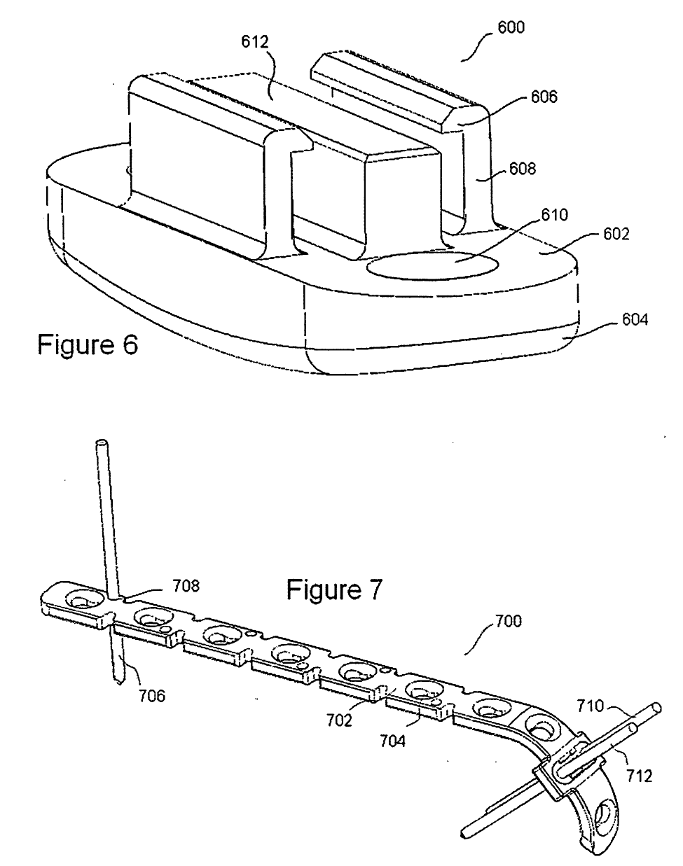

As shown in FIG. 4a (and with reference to FIGS. 5 and 6), the clamping fastener 414 comprises a cap 604 with smooth upper edges that is received within the well of the locking section. The cap has two through holes 421, 422, 610 in it, each one receiving a K wire 423. A lower surface of the cap has extending from it a rigid locking wedge 424 and a pair of locking tabs 425 that deflect longitudinally, slide into, and engage the parallel openings 420, 421. The interconnected holes 421, 422 in the locking section are in registry with the slanted lateral sides of the wedge (when it is inserted) such that the slanted sides 427 make contact with the K wires, at least when fully inserted. The slanted lateral edges of the wedge have a protruding gripping edge 426 located toward the bottom extent 428 of the wedge. When the fastener is fully inserted, each of the lateral gripping edges 426 serves to clamp a K wire between the wedge 426 and an opposite sidewall 450 of the closest through hole 421, 422 in the cap. Thus with the fastener fully inserted, the K wires are retained, the “snap” sensation delivered by the advancement of the tabs over the locking bars providing tactile or audible feedback to the surgeon that the fastener is locked in place. Note that the clamping and locking action of the fastener is accomplished by two separate mechanisms (wedge and tabs) that are functionally and mechanically separted from one another and that the deflection of the locking tabs and the clamping action of the wedge occur in different planes, particularly ones that are 90 degrees to one another.

As shown in more detail FIGS. 5 and 6, the clamping fastener 504 has two parallel tabs 512 with inwardly directed flanged tips 514 that deflect to pass over the locking bars and enter the openings 220, 221, snaping over the undersides of the cooperating locking bars 506 in the locking section. Note that the locking bars are located between the slot 214, 216 and the two interconnected openings 220, 221.

The plate of the present invention allows for the use of either screws or K wires for proximal fixation. FIG. 7 illustrates a lateral view of the olecranon plate with a K wire 706 inserted through a receiving hole thus fixing the plate to the ulnar shaft.

The olecranon plate of the present invention is anatomically specific for the fixation of olecranon fractures of the elbow. It is metal, for example, 316L stainless steel. It may use conventional dimensions for DCP screw holes, and provisional fixation holes.

Femoral Stem Cap

The femoral stem cap is a plastic protective cap that is placed over the femoral stem after the ball has been removed during total hip replacement surgery. The stem cap is round in cross section and internally tapered to fit snugly over the Morse taper of the femur. It may be externally tapered to reduce material consumption during manufacture. FIG. 1 illustrates a cutaway lateral view of the stem cap. The cap 800 has a sidewall 802 and a top wall 804. The top wall has a central round vent 806 that allows the escape of air while positioning the cap over the femoral stem. The interior of the cap 808 features a Morse taper that matches the taper on the femoral stem. The cap edge has a piloted opening 801 that allows for easy insertion and removal. The femoral stem cap is sized to fit the common sizes of Morse tapers used. It is made of plastic and is preferably sterile when applied.

We claim:

1. An olecranon plate, comprising:
a bone plate, having a curved portion adapted to the curvature of the olecranon,
the curved portion having formed in it a locking section for receiving a clamping fastener,
the locking section and fastener adapted to receive one or more K wires.

2. The olecranon plate of claim 1, wherein:
the locking section comprises a central slot that interconnects a pair of holes, the slot flanked by a pair of openings, the holes adapted to receive the one or more K wires;
the fastener comprising a cap in which is formed one or more apertures for receiving the K wires, each aperture being in registry with a hole in the locking section;
an underside of the cap having extending from it a pair of tabs that engage the pair of openings in the locking section.

3. The olecranon plate of claim 2, wherein:
the underside of the cap also comprising a a wedge with laterally slanting sides, the wedge adapted to enter the slot, at least one side having a gripping edge that is adapted to make contact with a K wire.

4. The olecranon plate of claim 3, wherein:
the gripping edge acts, when inserted into the slot, to clamp a K wire into the locking section.

5. The olecranon plate of claim 4, wherein:
the gripping edge acts on the K wire to clamp in between itself and a sidewall of a hole in the locking section that the K wire passes through.

6. The olecranon plate of claim 1, further comprising:
a tool for inserting the fastener, the tool comprising a shaft having at one end a handle and at the other end, a head;
the underside of the head adapted to cooperate with the fastener;
the head having a pair of through openings for receiving a pair of K wires, the shaft joining the head between the through openings.

7. The olecranon plate of claim 2, wherein:
the tabs have flanged ends that deflect and enter an opening prior to engaging a locking bar that is located between the opening and the slot.

8. The olecranon plate of claim 1, wherein:
the locking section is wider than the curved portion.

9. The olecranon plate of claim 1, wherein:
the K wires are two in number and spaced laterally from one another.

10. The olecranon plate of claim 9, wherein:
an underside of the clamping fastener has a locking wedge, with lateral gripping edges,
the wedge located between a pair of through holes that receive a pair of K wires.

11. A protective stem cap, comprising:
a plastic protective cap, generally round in cross section and internally tapered to fit snugly over a Morse taper.

12. The cap of claim 11, wherein:
the cap is externally tapered.

13. The cap of claim 11, wherein:
the cap has a sidewall and a top wall;

12. The cap of claim 13, wherein:
the top wall has a central vent that allows the escape of air while positioning the cap over a femoral stem.

13. The cap of claim 11, wherein:
the interior of the cap features a Morse taper that matches a taper on a femoral stem.

14. The cap of claim 11, wherein:
the cap has a free edge defining a piloted opening that allows for easy insertion and removal.

15. The cap of claim 11, wherein:
the cap is sterile.
